(19) United States
(12) Patent Application Publication
(10) Pub. No.: US 2009/0162374 A1
(43) Pub. Date: Jun. 25, 2009

(54) SPECIFIC REMOVAL OF ACTIVATED IMMUNE CELLS

(76) Inventors: Daniel E. Geraghty, Seattle, WA (US); Ni Lee, Mercer Island, WA (US)

Correspondence Address: MYERS BIGEL SIBLEY & SAJOVEC, PO BOX 37428, RALEIGH, NC, 27627, US

(21) Appl. No.: 12/064,537

(22) PCT Filed: Aug. 31, 2006
(86) PCT No.: PCT/US06/34033

§ 371 Date: Jul. 3, 2008

Related U.S. Application Data
(60) Provisional application No. 60/717,018, filed on Sep. 14, 2005.

(51) Int. Cl. A61K 39/395; G01N 33/53; A61K 35/14; A61K 35/12; C07K 16/00; A61P 37/06

(52) U.S. Cl. 424/153.1; 435/7.24; 424/529; 424/93.71; 530/388.73; 530/391.7

(57) ABSTRACT

The present invention provides a method of detecting an immune response in a mammalian subject by (a) withdrawing blood or a blood fraction containing immune cells from the subject, (b) contacting the blood with an antibody that specifically binds to the cell surface HLA-F histocompatibility protein of activated mammalian immune cells and then (c) detecting the presence or absence of binding of immune cells to the antibody, wherein the presence of binding indicates the presence of an immune response in the subject.

### RELATED APPLICATIONS

This invention claims the benefit of U.S. Provisional Patent Application Ser. No. 60/717,018, filed Sep. 14, 2005, the disclosure of which is incorporated by reference herein in its entirety.

This invention was made with Government support under Grant Numbers HD045813, AI33484 and AI49213 from the National Institutes of Health. The US Government has certain rights to this invention.

### FIELD OF THE INVENTION

This invention concerns methods and compositions for the treatment of autoimmune and alloimmune diseases.

### BACKGROUND OF THE INVENTION

A major genetic determinant of the immune response in general is contained within the major histocompatibility complex (MHC). In humans this region includes the classical class I loci HLA-A, -B and -C, The MHC contains in addition three highly homologous, non-classical class I genes, HLA-E, -F, and -G all three of which are located within the class I region and together with the classical class I antigens constitute the complete list of active class I genes in the human.

Recent work using new monoclonal antibodies reactive with HLA-F showed that while HLA-F was not surface expressed on most cell lines that contained intracellular protein, HLA-F was expressed on the surface of B cell and some monocyte cell lines and in vivo on extravillous trophoblasts that had invaded the maternal decidua (16, 23). On B LCLs, surface expression correlated with the presence some complex type N-glycosylated HLA-F present, although clearly not all the surface expressed HLA-F was fully glycosylated. Evidence of a physical association of HLA-F and TAP was reported (22), but surface expression was not reduced in TAP negative mutant lines (23). No peptide or other small ligand has been described as part of a mature HLA-F protein, but modeling of the structure suggested that the binding grove could support peptide ligand (25).

### SUMMARY OF THE INVENTION

A first aspect of the invention is a method of detecting an immune response in a mammalian subject. In general, the method is carried out by: (a) withdrawing blood or a blood fraction containing immune cells from the subject; (b) contacting the blood or blood fraction to an antibody (e.g., a monoclonal antibody) that specifically binds to the cell surface HLA-F histocompatibility protein of activated mammalian immune cells, which antibody does not bind to the cell surface of non-activated mammalian immune cells; and then (c) detecting the presence or absence of binding of the immune cells to the antibody, the presence of binding indicating the presence of an immune response in the subject. The detecting step may be carried out by any suitable technique, such as by heterogeneous immunoassay or by homogeneous immunoassay.

A further aspect of the invention is a composition comprising, consisting of or consisting essentially of a solid support having an antibody (e.g., a monoclonal antibody) coupled thereto, which antibody specifically binds to the cell surface HLA-F histocompatibility protein of activated mammalian T-lymphocytes, and which antibody does not bind to the-cell surface of non-activated mammalian T-lymphocytes.

A further aspect of the invention is a pharmaceutically acceptable composition comprising, consisting of or consisting essentially of a mammalian blood or blood fraction, the blood or blood fraction comprising blood serum, immune cells, optionally blood platelets, and optionally red blood cells, and wherein the immune cells (i) consist of non-activated immune cells that do not express HLA-F on the cell surface thereof, and (ii) are depleted of activated immune cells that express HLA-F on the cell surface thereof. Thus the composition is preferably depleted of activated immune cells activated by a preselected immunogen. In general, the composition may be produced by the process of: (a) withdrawing blood or a blood fraction containing immune cells from a mammalian subject afflicted with an undesired immune response; (b) contacting the blood or blood fraction to an antibody that specifically binds to the cell surface HLA-F histocompatibility protein of activated mammalian immune cells, which antibody does not bind to the cell surface of non-activated mammalian immune cells; and then (c) separating the blood or blood fraction from the antibodies to produce the composition.

A further aspect of the invention is a monoclonal antibody that specifically binds to the cell surface HLA-F histocompatibility protein of activated mammalian T-lymphocytes, and which antibody does not bind to the cell surface of non-activated mammalian T-lymphocytes. Preferably the antibody is a human monoclonal antibody or chimeric monoclonal antibody (the chimeric monoclonal antibodies having a human immunoglobulin constant region). In some embodiments the monoclonal antibody has a cytotoxic group coupled thereto. The monoclonal antibodies may be prepared as a composition or pharmaceutical formulation by combining them with a pharmaceutically acceptable carrier for administration to a patient.

A further aspect of the invention is a method of treating an undesired immune response in a subject in need thereof, comprising administering the subject a monoclonal antibody as described herein in an amount effective to treat the disease.

Suitable immune cells in the invention described herein include but are not limited to T-lymphocytes, B-lymphocytes, NK cells, monocytes, and combinations thereof.

In some embodiments of the invention the immune response is caused by an infection; in some embodiments the immune response is an autoimmune disease; in some embodiments the immune response is an allergic response or alloimmune disease (e.g., a graft versus host disease or a tissue transplant rejection).

Preferably, antibodies of the invention or used in carrying out the invention do not bind to the HLA-A, HLA-B, HLA-C, HLA-E, or HLA-G histocompatibility proteins of activated mammalian T-lymphocytes in either activated or non-activated form.

An advantage of the present invention is that HLA-F is only expressed on the cell surface of activated immune cells such as activated CD4 and CD8 T cells, and not on the surface of cells such as CD4+ CD25+ regulatory T cells, nor on the surface of CD21, CD33, or CD44 cells. Hence these latter cells are not bound, removed and/or depleted by the methods of the present invention as they are with, for example, by methods utilizing anti-CD25+ antibodies. See, e.g., Powell et al., Large scale depletion of CD25+ Regulatory T cells from Patient leukapheresis samples, J. Immunotherapy 28(4) (July/August 2005)).

The foregoing and other objects and aspects of the present invention is explained in greater detail in the drawings herein and the specification set forth below.

### BRIEF DESCRIPTION OF THE DRAWINGS

FIG. 1. HLA-F surface expression is upregulated on the surface of activated PBMC subsets without any corresponding increase in overall cellular protein levels. A) FACS profiles were generated using double staining with each of the anti-F antibodies 4A11 (solid line) and 3D11 (dashed line), the anti-HLA-E reagent 3D12 (dotted line), and control mouse IgG1 (shaded histogram) combined with reagents specific for each of CD19 (B cells), CD4 (T helper cells), CD8 (cytolytic T cells), and CD56 (NK cells) as outlined in methods. Representative profiles are shown before activation (day 0) and 3-5 days after activation as indicated. B) Western analysis of the same cell populations before and after activation with timing identical to that for the FACS analysis. Cell subtypes are indicated above and below the respective lane for pre-activated and activated cells respectively. Monocytes isolated via CD14 were included in western analysis only.

FIG. 2. Kinetics of HLA-F expression on CD3+CD8+ T cells after activation. HLA-F surface expression is upregulated as early as 3 hours after T cell activation and increases linearly over 18 hours. The six histograms present FACS analysis of a time course monitoring HLA-F expression on the surface of T cells isolated from peripheral blood and activated with PMA/ionomycin using anti-F antibodies 4A11 (solid line), 3D11 (dashed line), and mouse IgG1 (shaded). Differential staining of the two anti-F antibodies is evident over the time course possibly indicating different protein forms of HLA-F are expressed with different kinetics as discussed in the text.

FIG. 3. HLA-F surface expression on T cell clones is upregulated after stimulation and downregulated upon resting (that is, are “non-activated”). Upper: HLA-F expression was measured on the surface of a representative T cell clone stimulated by anti-CD3 antibody after 8, 12, and 15 days as indicated above each histogram. Cells were then rested for 7 days and tested again for HLA-F expression (indicated above rightmost profile). FACS profiles were generated by staining with each of the anti-F antibodies 4A11 (solid line) and 3D11 (dashed line), the anti-HLA-E reagent 3D12 (dotted line), and control mouse IgG1 (shaded histogram). Lower: HLA-F is expressed predominantly in an Endo H sensitive form on the surface of activated T cell clones. CD3+CD8+ T cell clones were collected at day 13 after stimulation. Cell surface protein was biotinylated and cell lysate was immunoprecipitated with streptavidin-agarose beads. The avidin-bound immunocomplexes were treated (+) or untreated (−) with Endo H, separated in 11% SDS-PAGE and analyzed by Western blot using anti-HLA-F mAb 3D11. Surface HLA-F proteins were detected on B-LCL cell line 721.221 and monocyte cells 90196B to demonstrate the control for Endo H digestion. HLA-F in total cell lysate of B-LCL line TM previously analyzed (23) was analyzed as an additional control.

FIG. 4. HLA-F surface expression presents altered kinetics but not expression levels on TAP and Tapasin (TPN) negative cells in vivo. PBMC from a TAP negative individual (34) a Tapasin negative individual (35), and a normal control was activated with PMA plus ionomycin. FACS profiles were generated using each of the anti-F antibodies 4A11 (solid line) and 3D11 (dashed line), the anti-HLA-E reagent 3D12 (dotted line), and control mouse IgG1 (shaded histogram). Cells were analyzed on days 1, 2 and 5 after stimulation as indicated above the profiles (Tapasin-cells were only analyzed on days 1 and 2). Cell subsets analyzed are indicated to the left of each pair of profiles and the source of the PBMC is indicated to the upper left of each set of FACS profiles as TAP⁻, TPN⁻ or PBMC indicating normal control cells.

FIG. 5. Antigen specific activation of T cells induces HLA-F surface expression. A) Upper PBMC isolated from CMV+individual with HLA-A2 haplotype stimulated by mature DCs loaded with CMV peptide pp65 were stained with anti-CD25 reagent (vertical) and anti-CD3 antibody (horizontal) to demonstrate antigen specific activation of CMV reactive T cells as previously described (28). Lower T cell subsets activated (CD3+. CD25+) and not activated (CD3+, CD25⁻ and circled in upper were simultaneously stained with each of the anti-F antibodies 4A11 (solid line) and 3D11 (dashed line), the anti-HLA-E reagent 3D12 (dotted line), and control mouse IgG1 (shaded histogram). Surface HLA-F was evident only on T cells expressing the IL-2 receptor alpha chain marker CD25. B) Upper Unstimulated PBL isolated from HLA-A2 individual tested for CD25+ CD4+ T regulatory cells. Lower T regulatory cell subsets (circled in upper) were simultaneously stained with 4A11 (solid line) and 3D11 (dashed line), the anti-HLA-E reagent 3D12 (dotted line), and control mouse IgG1 (shaded). HLA-F expression was completely absent on this cell subset.

FIG. 6. Depletion of HLA-F expressing cells after primary MLR inhibits secondary MLR without compromising third party reactivity.

FIG. 7. Amino acid sequences of human HLA-F and non-human primate homologous proteins. Mamu-F, Macaca mulatta-F; Mane-F, Macaca nemestrina-F; Mafa-F, Macaca fascicularis-F; Paca-F, Papio cynocephalus anubis-F.

FIG. 8. HLA-F equivalent proteins found in 4 nonhuman primates (NHP) are reactive with human anti-HLA-F antibodies and expression patterns of NHP-F proteins parallel those found in humans. Mamu, Macaca mulatta; Mane, Macaca nemestrina; Mafa, Macaca fascicularis; Paca, Papio cynocephalus anubis.

### DETAILED DESCRIPTION OF THE PREFERRED EMBODIMENTS

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all United States patents cited herein are to be incorporated herein by reference in their entirety.

### A. DEFINITIONS

“Histocompatibility proteins” of the major histocompatibility complex (MHC) are known. In humans this region includes the classical class I loci HLA-A, -B and -C, whose role in immunological recognition is now well understood (1). These proteins bind and present peptide to cytotoxic T cells and interact with receptors on natural killer (NK) cells to inhibit lytic activity or cytokine production (2-4). Intracellular transport and cell surface expression of class I proteins are dependent on the availability of peptides within the endoplasmic reticulum actively transported there by the transporter associated with antigen processing (TAP) complex (5, 6). The MHC contains in addition three highly homologous, non-classical class I genes, HLA-E, -F, and -G all three of which are located within the class I region and together with the classical class I antigens constitute the complete list of active class I genes in the human (7-10). Each of the non-classical class I genes can be distinguished from classical class I by their expression patterns, and for HLA-E and HLA-G with respect to peptide binding properties and function. HLA-E ubiquitously expressed but at much lower relative levels than the classical molecules (8, 11), and HLA-G is specifically expressed in placental tissue (12-14). HLA-G binds a relatively narrow range of peptides probably serving as structural components rather than for antigen presentation (15, 16) while HLA-E complexes with nonoamer peptide derived from the signal sequence of other MHC class I, including HLA-A, B, C, and G but excluding HLA-F (17). The function of HLA-G is not completely clear but may act as an inhibitory ligand by interacting with the ILT2 and ILT4 receptors (18). HLA-E complexed with nonamer peptide from other MHC class I interacts with the lectin receptors CD94 combined with different NKG2 subunits to inhibit and activate primarily NK cells (11, 19). HLA-E expressed in the placenta and complexed with nonamer derived from the HLA-G signal sequence may have a unique function there (16, 20). The function of HLA-F is unknown but tissue and cell specific mRNA expression was observed (9) and protein expression has largely correlated with that (16, 21-23). The HLA-F locus has very little allelic polymorphism and is highly conserved in distantly related nonhuman primates (24) suggesting a conserved function.

“Immune Response” generally refers to inate and acquired immune responses, including but not limited to both humoral immune responses (mediated by B lymphocytes) and cellular immune responses (mediated by T lymphocytes).

“Undesired Immune response” as used herein refers to an immune response that results in a pathological condition in the subject such as an autoimmune disease, alloimmune disease, allergic reaction, a decoy immune response resulting from an infection, etc.

“Acquired immune deficiency disease” as used herein includes but is not limited to that caused in humans by HIV, as well as in monkeys by SIV and cats by FIV.

“Autoimmune diseases” are generally believed to be caused by the failure of the immune system to discriminate between antigens of foreign invading organisms (non-self) and tissues native to its own body (self). When this failure to discriminate between self and non-self occurs and the immune system reacts against self antigens, an autoimmune disorder may arise. Autoimmune diseases, or diseases having an autoimmune component, include multiple sclerosis, rheumatoid arthritis, juvenile oligoarthritis, type I diabetes mellitus, inflammatory bowel disease, Crohn's disease, scleroderma, psoriasis, atherosclerosis, Hashimoto's thyroiditis, Addison's disease, and systemic lupus erythematosus (SLE). See, e.g., U.S. Pat. Nos. 6,894,038; 6,890,954; and 6,800,300.

“Alloimmune diseases” are referred to herein as disorders such as graft versus host disease and tissue transplant rejection, in which an immune response against or by foreign, transplanted tissue can lead to serious complications or be fatal. The foreign tissue may be from the same species or, in the case of a xenograft, from a different species (such as pig tissue transplanted into a human). In the treatment of these disorders, it is desired to prevent the body from reacting against non-self antigens. See, e.g., U.S. Pat. No. 6,800,300.

“Allergy” or “allergic response” as used herein includes an immune response sensitivity to a normally harmless substance, or “allergen”, that does not bother most people. Such allergens include but are not limited to venom (including insect venoms such as bee and wasp venom and reptile venoms such as snake venom), animal dander, pollen (including pine pollen, ragweed pollen, etc.), fungi or fungal spores, dust mites, foods allergens (e.g., milk, egg, peanut, tree nuts such as walnut and cashew, fish, shellfish, soy, and wheat), drugs (e.g., antibiotics (for example, penicillins such as penicillin, ampicillin, and amoxil, cephalosporines such as Keflex, Cefzil, Ceftin, Suprax, and Vantin; Sulfas such as Septra, Bactrim, and Pediazole, anticonvulsants such as Dilantin, Tegretol, Depakene, and Lamictal, and insulin such as pork and beef insulins). “Allergy” or “allergic response” as used herein also includes allergic contact dermatitis in response to chemicals such as urushiols in plants such as poison ivy, poison oak and poison sumac.

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be humanized or chimeric antibodies. The term “antibody” as used herein includes antibody fragments which retain the capability of binding to a target antigen or receptor, for example, Fab, F(ab′)₂, Fc and Fv fragments, and the corresponding fragments obtained from antibodies other than IgG. Such fragments are also produced by known techniques.

“Infection' as used herein refers to the pathological state resulting from the invasion of the body by pathogenic microorganisms and includes viral infections, bacterial infections, fungal, protozoal and parasitic infections, etc. See, e.g., U.S. Pat. No. 6,902,743. Examples include but are not limited to toxic shock syndrome (Staphylococcus, Streptococcus), meningitis (both bacterial and viral/Group B Streptococcus, Escherichia coli, Listeria monocytogenes, Streptococcus pneumoniae (pneumococcus) and Neisseria meningitides and septicemia. Autoimmune diseases such as multiple sclerosis may be triggered by an infection.

“Viral infection” as used herein means an infection due to a DNA virus or an RNA virus (retrovirus). Examples of a double-stranded DNA virus are the Herpes virus and the influenza virus. Human immunodeficiency virus (HIV) is a prototype for retroviruses, i.e., viruses that use reverse transcription to replicate. However, certain DNA viruses use, in part, reverse transcription mechanisms to replicate such as, for example, the Hepatitus B virus.

“Viruses” include retroviruses such as HIV or HTLV, influenza, rhinoviruses, herpes, hepatitis, or the like.

“Bacterial infection” as used herein includes but is not limited to infections caused by one of more of gram positive or gram negative bacteria, examples of which include but are not limited to Staphylococcus aureus, Streptococcus pyogenes, Streptococcus viridans, Enterococcus, anaerobic Streptococcus, Pneumococcus, Gonococcus, Meningococcus, Mima, Bacillus anthracis, C. diphtheriae, List. monocytogenes, Streptobacillus monohiliformis, Erysipelothrix insidiosa, E. coli, A. aerogenes, A. faecalis, Proteus mirabilis, Pseudomonas aeruginosa, K. pneumoniae, Salmonella, Shigella, H. influenzae, H. ducreyi, Brucella, Past. pestis, Past. tularensis, Past. muiltocida, V. comma, Actinobacillus mallei Pseud. pseudomallei, Cl. tetani, Bacteroides, Fusobacterium fusiforme, M. tuberculosis, atypical niycobacteria, Actinomyces israelii, Nocardia, T. pallidum, T. pernue, Borrelia recurrentis, Peptospira, Rickettsia, and Mycoplasina pneumoniae.

“Fungal, protozoal, and parasitic infections” as used herein include those caused by organisms such as Cryptococcus neoformans, Histoplasma capsulatum, Candida albicans, Candida tropicalis, Nocardia asteroides, Rickettsia ricketsil, Rickettsia typhi, Mycoplasma pneumoniae, Chlamydial psittaci, chlamydial trachomatis, plasmodium falciparum, Trypanosoma brucei, Entamoeba histolytica, Toxaplasma gondii, Trichomonas vaginalis, Schistosoma mansoni, and the like.

“Immune cells” with which the present invention is concerned include peripheral blood mononuclear cells (PBMCs) in general, the activated counterparts thereof (which are not necessarily mononuclear) and the corresponding cells when not peripheral but resident in an organ or tissue such as spleen, gut, lymph nodes, etc. Immune cells that, when activated, express the HLA-F histocompatibility protein on the cell surface (or “extracellular surface”), thereof are of particular interest. Examples of immune cells with which the present invention is concerned include but are not limited to T-lymphocytes, B-lymphocytes, natural killer (NK) cells, and monocytes. The present invention is generally not concerned with immune cells on which HLA-F is not expressed, such as dendritic cells, neutrophils, eosinophils, and basophils.

“Activated” immune cells are those that have been induced (typically by contact to an immunogen) to perform an immune response function that does not occur in the non-activated immune cell prior to such activation. Such an immune response function may be proliferation, cytolysis, cytokine production and/or secretion, immunoglobulin production and/or secretion, etc., including combinations thereof. As disclosed herein, activated immune cells are typically characterized by the presence of HLA-F on the extracellular surface thereof, while non-activated immune cells are typically characterized by the absence of HLA-F on the extracellular surface thereof (e.g., as detectable by anti-HLA-F antibodies as described herein). However, and as noted above, an important advantage of the present invention is that regulatory T cells such as CD4+ CD25+ regulatory T cells do not express HLA-F on the surface thereof, and are not consequently are not bound or removed by antibodies of the present invention.

“Solid support” as used herein refers to an inert material or molecule to which an antibody may be bound or coupled, either directly or indirectly through a linking group. The solid support is suitable for use in column chromatography or other types of purification. For diagnostic immunoassays solid supports may be the same as or different from the types of solid supports used for chromatography, with suitable examples including but not limited to beads (including but not limited to magnetic beads such as described in U.S. Pat. No. 6,867,041, U.S. Pat. No. 5,858,358 and U.S. Pat. No. 6,905,874), plates, slides or wells formed from materials such as latex or polystyrene, etc.

“Subjects” to be treated by the present invention are primarily human subjects (including male and female subjects and neonatal, pediatric, adolescent, adult and geriatric subjects), but the invention may also be carried out on animal subjects, particularly mammalian subjects such as mice, rats, companion animals such as dogs and cats, livestock and horses for veterinary purposes, and for drug screening and drug development purposes.

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms.” to any type of treatment that imparts a benefit to a patient afflicted with a disease, including improvement in the condition of the patient (e.g., in one or more symptoms), delay in the progression of the disease, etc.

“Cytotoxic agent” as used herein includes but is not limited to ricin (or more particularly the ricin A chain), aclacinomycin, diphtheria toxin. Monensin, Verrucarin A, Abrin, Vinca alkaloids, Tricothecenes, and Pseudomonas exotoxin A.

### B. ANTIBODIES

Antibodies and the production thereof are known. See, e.g., U.S. Pat. No. 6,849,719; see also U.S. Pat. Nos. 6,838,282; 6,835,817; 6,824,989.

Antibodies of the invention include antibodies that are modified, i.e, by the covalent attachment of any type of molecule to the antibody such that covalent attachment does not prevent the antibody from specifically binding to its binding site. For example, antibodies of the invention may be modified, e.g., by glycosylation, acetylation, pegylation, phosphylation, amidation, or with other protecting/blocking groups, proteolytic cleavage, linkage to a cellular ligand or other protein, etc. Any of numerous chemical modifications may be carried out by known techniques, including but not limited to specific chemical cleavage, acetylation, formylation, metabolic synthesis of tunicamycin, etc. Additionally, the antibodies may contain one or more non-classical amino acids.

Polyclonal antibodies of the invention can be generated by any suitable method known in the art. For example, a suitable antigen can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

Monoclonal antibodies can be prepared using a wide variety of techniques including the use of hybridoma, recombinant, and phage display technologies, or a combination thereof. For example, monoclonal antibodies can be produced using hybridoma techniques including those taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and known. Briefly, mice are immunized with an antigen or a cell expressing such antigen. Once an immune response is detected, e.g., antibodies specific for the antigen are detected in the mouse serum, the mouse spleen is harvested and splenocytes isolated. The splenocytes are then fused by known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain.

For example, antibodies can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include but are not limited to those disclosed in U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art.

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (see, e.g., U.S. Pat. Nos. 5,225,539; 5,530,101; and 5,585,089), veneering or resurfacing (see, e.g., EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are desirable for therapeutic treatment, diagnosis, and/or detection of human patients. Human antibodies can be made by a variety of methods known in the art including phage display methods described above using antibody libraries derived from human immunoglobulin sequences. See, e.g., U.S. Pat. Nos. 4,444,887 and 4,716,111.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,413,923; 5,625,126; 5,633,425; 5,569,825; 5,661,016; 5,545,806; 5,814,318; and 5,939,598.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

### C. SOLID SUPPORTS AND ANTIBODIES COUPLED TO SOLID SUPPORTS

Any suitable solid support may be used in the present invention. Solid supports include inorganic materials, organic materials, and combinations thereof. Examples of suitable solid support materials include membranes, semi-permeable membranes, capillaries, microarrays, multiple-well plates comprised of alumina, alumina supported polymers, polysaccharides including agarose, dextran, cellulose, chitosan, and polyacrylamide, polyacrylate, polystyrene, polyvinyl alcohol, glass, silica, silicon, zirconia, magnetite, semiconductors and combinations thereof. The solid support material may be in the form of magnetic, typically spherical, beads such as described in U.S. Pat. No. 6,867,041, U.S. Pat. No. 5,858,358 and U.S. Pat. No. 6,905,874. The support may be particulate or divided form having other regular or irregular shapes or it may be in the form of. Preferred solid support materials are those having minimal non-specific binding properties and that are physically and chemically resistant to the conditions used in the purification process employed in this invention such as changes in pH and ionic strength.

Antibodies may be bound to solid support resin either directly or indirectly. When bound directly the antibody is coupled to the solid support material by formation of covalent chemical bonds between particular functional groups on the antibody (e.g., primary amines, sulfhydryls, carboxylic acids, hydroxyls, and aldehydes) and reactive groups on the support. A variety of activating compounds and schemes for directly bonding compounds to solid phase supports are known in the art (See U.S. Pat. No. 6,555,391, U.S. Pat. No. 6,773,599). Some examples of such activating compounds include cyanogen bromide, cyanuric chlorde, epichlorohydrin, divinyl sulphone, p-toluenesulphonyl chloride, 1,1′-carbonyldiimidazole, sodium meta-periodate, 2-fluoro-1-methylpyridiniumtoluene-4-sulphonate, glycidoxypropyltrimethoxysilane and 2,2,2-trifluoroethanesulphonyl chloride. The procedures by which such activating steps are carried out are known to those skilled in the art.

The solid support may be contained within a column or cartridge and in any suitable form, and packaged in a sterila package to provide a product suitable for installing or inserting in an extracorporeal blood processing apparatus as discussed generally below. See, e.g., U.S. Pat. No. 6,773,591.

### D. DIAGNOSTIC IMMUNOASSAYS

Diagnostic immunoassays may be carried out by any suitable technique, including heterogenous assays and homogenous assays. See, e.g., U.S. Pat. Nos. 6,897,030; 6,884,591; 5,599,678; and 5,200,346; see also E. Maggio, Enzyme-Immunoassay, (1980)(CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” In some assays the antibody may be labelled with a detectable group immobilized on a solid support, depending upon the assay format. Numerous variations are known and readily apparent to those skilled in the art.

Subjects for such assays may be human or animal subjects afflicted with, or suspected of being afflicted with, an undesired immune response such as an autoimmune disease or alloimmune disease as described herein, or an allergic reaction to an allergen.

For example, biological sample, blood or blood fraction containing PBMCs may be withdrawn from the subject, contacted to an antibody of the invention immobilized on a solid support, and then the solid support contacted to an antibody of the invention having a detectable group coupled thereto in a “sandwich” assay, where the presence of the detectable group on the solid support (typically after a rinsing step) indicates the presence of activated PBMCs, and hence an undesired immune response, in the subject.

Antibodies as described herein may be conjugated to a solid support suitable for a diagnostic assay in accordance with known techniques, such as precipitation. Antibodies as described herein may likewise be conjugated to detectable groups in accordance with known techniques.

### E. EXTRACORPOREAL TREATMENT OF IMMUNE DISORDERS

A further aspect of the invention is a method of treating an undesired immune response in a mammalian subject in need thereof. In general, the method is carried out by (a) withdrawing blood or a blood fraction containing immune cells from the subject; (b) contacting the blood or blood fraction to an antibody as described herein (e.g., one that specifically binds to the cell surface HLA-F histocompatibility protein of activated mammalian immune cells, which antibody does not bind to the cell surface of non-activated mammalian immune cells); (c) separating the blood or blood fraction from the antibodies to thereby deplete the blood or blood fraction of activated immune cells; and then (d) returning the blood or blood fraction to the subject. In general, the antibody can be coupled to a solid support as described above. Patients or subjects that may be treated include those in which the immune response is caused by an infection, by an autoimmune disease by an allergic response, an alloimmune disease, graft versus host disease, tissue transplant rejection, etc.

Techniques for extracorporeal blood processing are well known and can be readily adapted to implement the present invention. See, e.g., U.S. Pat. Nos. 6,902,539; 6,830,553; 6,811,749; 6,790,195; 6,773,413; 6,764,460; 6,730,233; 6,730,055; 6,691,040; 6,537,445; 6,284,142; 4,077,882; 3,963,023; and 3,946,731. The withdrawing step (a) and the returning step (d) can be carried out continuously or in batch form in accordance with known techniques. For example, in some embodiments steps (a) through (d) are carried out by apheresis in accordance with known techniques.

### F. BONE MARROW TRANSPLANT PROCEDURES

A further aspect of the invention is a method of carrying out an allogenic bone marrow transplant in a subject in need thereof. Suitable subjects include, but are not limited to, those afflicted with leukemia, severe aplastic anemia, lymphoma, multiple myeloma, immune deficiency disorder, or solid tumor cancer.

In some respects the invention provides a method for the prevention of or prophylaxis against graft versus host disease (GVHD) in a patient to undergo a bone marrow transplant, (preferably where bone marrow of an allogenic donor has been matched to the patient for HLA compatibility), comprising the steps of treating the bone marrow of the donor with one or more antibodies of the present invention (e.g., immobilized on a solid support) in an amount sufficient to deplete activated immune cells therefrom, and then transplanting the treated bone marrow to the patient. Such treatments can be carried out in accordance with known techniques, modified to utilize the antibodies described herein. See, e.g., U.S. Pat. Nos. 6,649,189; 6,387,366; 5,576,085; 5,593,677; 4,486,188; and 4,481,946.

In some embodiments the procedures involve: (a) providing a first cell preparation comprising blood stem cells and immune cells; (b) contacting the first cell preparation to at least one isoantigen from the subject to activate a portion of the immune cells; then (c) contacting the cell preparation to an antibody that specifically binds to the cell surface HLA-F histocompatibility protein of activated mammalian T-lymphocytes, which antibody does not bind to the cell surface of non-activated mammalian T-lymphocytes; then (d) separating the cell preparation from the antibodies to produce a depleted cell preparation depleted of activated immune cells; and then (e) administering the depleted cell preparation to the subject in a bone marrow transplant-effective amount, wherein the cell preparation is depleted of immune cells activated by the isoantigen which would otherwise produce autoantibodies against the subject but contains non-activated immune cells effective for producing an immune response against non-self immunogens by the subject.

The cell preparation of step (a) may be prepared by any suitable technique, such as bone marrow harvest or by apheresis of peripheral stem cells. In some embodiments, the cell preparation of step (a) is collected from a haploid identical (or “haploidentical”) donor. In other embodiments the cell preparation of step (a) is collected from a mismatched unrelated donor.

The “isoantigen” for use in contacting step “b” may be any suitable cells or cell fragments derived from cells of the patient, including cell membranes. The contacting step may be carried out in vitro in accordance with standard techniques. The contacting and separating steps step “c” and “d” may be carried out in batch form or continuously in like manner as discussed above in connection with extracorporeal blood processing. The depleted cell preparation may likewise be administered in step “e” in accordance with known techniques carried out in bone marrow transplantation.

An example protocol in which the invention can be carried out is as follows:

### Example Protocol Objectives

A. Primary Objectives. Assess the safety of TBI, Thiotepa and Fludarabine regimen without ATG for engraftment of haploidentical CD34+ selected peripheral blood stem cells.

B. Secondary Objectives. (1) Examine the risk for severe graft-vs.-host disease; (2) Examine the kinetics of immune reconstitution; (3) Examine the risk for life-threatening infections.

### Example Protocol Patient Selection

A. Inclusions. Patients with life-threatening hematologic malignancies including: Acute leukemia advanced beyond first remission; Acute leukemia in first remission with very high-risk prognostic features (Ph+ ALL, 11q23 abnormality AML or ALL, hypodiploid ALL, failure to achieve first remission within 1 month after induction, secondary AML). Patients with first remission must be approved by PCC; Myelodysplastic syndromes with International Prognostic Score; Chronic myelogenous leukemia in accelerated or blast phase. Age <21 years.

B. Exclusions Patients with a suitable HLA-matched related or unrelated donor. Patients with a related donor mismatched for a single HLA-A, B, C, DRB1, or DQB1 antigen. Patients who are HIV+ due to high risk of viral reactivation. Patients who are pregnant. Organ dysfunction; Cardiac ejection fraction <45%.; Severe pulmonary dysfunction (DLCO <60% of predicted); Liver function tests including total bilirubin, SGPT and SGPT 2× the upper limit of normal unless caused by the malignancy; Patients with a life expectancy <6 months from coexisting disease other than the malignancy. Patients undergoing second marrow transplant, after a first regimen containing TBI

### Example Protocol Donor Selection

A. Inclusions. Haploidentical family members. Preference will be given to the donor with the least degree of HLA-mismatch including HLA-A, B, C, DRB1, and DQB1. NK inhibitory receptor (KIR) preference. Among equally mismatched donors, the priority will be given to a donor with expression of a KIR ligand from a ligand group that is not expressed by the patient. Donor must have adequate veins for leukapheresis or agree to placement of central venous catheter (femoral, subclavian).

B. Exclusions. Donors who for psychologic, physiologic or medical reasons are unable to tolerate PBSC collection. Age <18 years. Donors who are HIV+ due to high risk of viral reactivation. Female donors of childbearing years must have a negative pregnancy test.

### Example Protocol Plan of Treatment

### A. Overall Treatment Schema

Age > 7 years

| | Day Relative to Transplant | | | | | | | | | |
|---|---|---|---|---|---|---|---|---|---|---|
| Treatment | −7 | −6 | −5 | −4 | −3 | −2 | −1 | 0 | 1 | 2 |
| Donor | | | | | | | | | | |
| G-CSF | | | | X | X | X | X | X | X | X |
| Apheresis | | | | | | | X | X | X | X |
| Patient | | | | | | | | | | |
| TBI (8 Gy) | X | | | | | | | | | |
| Fludarabine (40 mg/m2/ | | X | X | X | X | X | | | | |
| day) | | | | | | | | | | |
| Thiotepa (5 mg/kg × 2) | | | X | | | | | | | |
| CD34+ Cell Infusion | | | | | | | | X | | X |

Age ≦ 7 years

| | Day Relative to Transplant | | | | | | | | | | | |
|---|---|---|---|---|---|---|---|---|---|---|---|---|
| Treatment | −9 | −8 | −7 | −6 | −5 | −4 | −3 | −2 | −1 | 0 | 1 | 2 |
| Donor | | | | | | | | | | | | |
| G-CSF | | | | | | X | X | X | X | X | X | X |
| Apheresis | | | | | | | | | X | X | X | X |
| Patient | | | | | | | | | | | | |
| TBI (2 Gy | X | X | X | | | | | | | | | |
| BID) | | | | | | | | | | | | |
| Fludarabine | | | | X | X | X | X | X | | | | |
| (40 mg/m2/ | | | | | | | | | | | | |
| day) | | | | | | | | | | | | |
| Thiotepa | | | | | X | | | | | | | |
| (5 mg/kg × 2) | | | | | | | | | | | | |
| CD34+ Cell | | | | | | | | | | X | | X |
| Infusion | | | | | | | | | | | | |

### B. Conditioning Regimen

1. Central Nervous System Prophylaxis: Patients with acute lymphoblastic leukemia will receive intrathecal methotrexate twice before the transplant and four times after day 35. Patients with other hematologic malignancies will have a diagnostic lumbar puncture before the preparative regimen. If the CSF is positive for malignant cells, the patient will receive intrathecal methotrexate.

2. TBI. Patients >7 years of age: TBI will be given on day −7 as a single 8 Gy dose at a rate of 16 cGy/minute. The dose to the lungs will be limited to 4 Gy by use of lung shields. Patients ≦7 years of age: TBI will be given on days −9, −8, and −7 twice daily at a dose of 2 Gy for a total dose of 12 Gy. The dose to the lungs will be limited to 9 Gy by use of lung shields.

3. Fludarabine—Fludarabine will be given at a dose of 40 mg/m²/day IV on days −6, −5, −4, −3, and −2 for a total dose of 200 mg/m².

4. Thiotepa—Thiotepa will be given on day −5 at in two doses of 5 mg/kg IV separated by 12 hours, for a total dose of 10 mg/kg. The dose should be calculated using the actual body weight or the adjusted ideal body weight, whichever is lower.

### C. Collection and Infusion of CD34+ Selected PBSC

1. Treatment Schema for the Donor

| | Days | | | | | | |
|---|---|---|---|---|---|---|---|
| | −4 | −3 | −2 | −1 | 0 | 1 | 2 |
| G-CSF 16 μg/kg | X | X | X | X | X | X | X |
| S.C. | | | | | | | |
| Leukapheresis | | | | X | X | X | X |

2. G-CSF Administration to Donors: All donors will receive G-CSF 16 mg/kg/day SC for a minimum of 5 consecutive days, beginning on day −4. G-CSF doses will be administered before 11:00 a.m. each day. The schedule of G-CSF administration and PBSC collections can only be ascertained once the treatment regimen has been established by the coordinator and day 0 identified.

3. PBSC Collection: Donors may undergo vein to vein collections or, if PBSC cannot be collected by a vein to vein technique, a percutaneous Mahurkar catheter will be inserted. PBSC will be collected on day −1 and day 0. The product collected on the afternoon of day −1 and stored in the refrigerator at 4° C. overnight. The second PBSC collection will be performed on the morning of Day 0 and pooled with the Day −1 collection. The pooled product will be processed for CD34 enrichment and the selected cells infused on day 0. If the final product on day 0 contains less than 10.0×10⁶ CD34+ cells per kg recipient weight, a third collection will be performed on Day 1 (stored overnight) and pooled with a fourth collection from Day 2, processed for CD34 enrichment, and infused on Day 2. General procedures will include the use of a standard apheresis machine (COBE Spectra, Lakewood Colo.), and processing up to 16 liters of whole blood during the collection.

4. CD34+ Selection: Selection of CD34+ cells will be according to the procedure outlined in Appendix C. If, after the first CD34+ selection procedure the total number of CD34+ cells is less than 10×10⁶/kg recipient weight, then a second CD34+ selection procedure will be performed on the combined apheresis products obtained from days +1 and +2. The total number of CD3 cells administered to the patient will not be greater than 1×10⁴/kg recipient weight. There will be a minimum of 5.0×10⁶ CD34+ cells/kg recipient weight. In the instance that these limits are mutually exclusive and cannot be achieved with further graft manipulation, the donor product will contain a minimum of 7.5×10⁶ CD34+ cells/kg recipient weight and the upper limit to numbers of CD3 cells infused will be lifted.

5. CD34+ Cell Infusion: CD34+ selected cells will be infused through a central venous catheter on day 0 of the treatment regimen; a second infusion may be given on day +2, as noted above. Refer to Standard Practice Manual for guidelines regarding infusion of peripheral blood stem cells.

There will be no post-transplant immunosuppression since GVHD prophylaxis will be provided by CD34+ selection/T-cell depletion of the stem cell product. Patients are not eligible to receive post-transplant growth factors for 21 days.

### Example Protocol Infection Prophylaxis

1. Immunoglobulin (IVI g; 500 mg/kg) should be administered to all patients every other week during marrow transplantation, and every three weeks after discharge (or after day 100). IVIg shall continue until lymphoid function is restored, as demonstrated by adequate response to immunization with hepatitis, tetanus, pneumococcus, and inactivated polio vaccines.

2. All patients will receive prophylaxis for pneumocystis carinii (PCP) prior to HSCT. Bactrim or alternate agent should be initiated for those not already receiving prophylaxis. PCP prophylaxis should be discontinued 48 hours prior to marrow infusion (day −2). PCP prophylaxis should be resumed at day 21 following marrow infusion, provided ANC >500, and continued until day +3650R 6 months after completion of immunosuppressant therapy given for GVHD, whichever comes later. For patients with allergy to Bactrim, refer to Standard Practice Manual for guidelines regarding desensitization therapy and use of other agents for PCP prophylaxis.

3. Antifungal prophylaxis—Patients should receive antifungal prophylaxis from day +1 through day +100 after transplant. Prophylaxis may include one of the following: a) ABLC (5 mg/kg/day)—for all children ≦12 years old; alternative for renal impairment is Ambisome (5 mg/kg/day); or b) Voriconazole—reserved for patients >12 years old. Pretransplant patients with ANC <500 should commence Ambisome prophylaxis on arrival to the center.

4. HSV prophylaxis—HSV seropositive patients should receive high dose acyclovir (500 mg/m2 TID) day −11 through day +28. Valacyclovir (1 gm po TID or 500 mg po TID if <40 kg) may be used for patients able to tolerate oral medication. After day 28, decrease dose interval to twice daily.

5. CMV Monitoring and Prophylaxis—

a. Pre-transplant: All patients should be evaluated with CMV antigen studies and plasma PCR (virology lab). Pre-transplant patients who are positive for CMV by PCR or have antigenemia ≧5 cells/slide should be treated with induction Ganciclovir (GCV) at 5 mg/kg IV twice daily for 7 days, followed by 5 mg/kg/day IV for 5 days prior to initiation of conditioning regimen.

b. Post-transplant: i. All patients should be monitored weekly for CMV activation by

CMV antigen studies (day +10 through +100 after conditioning) and plasma PCR (day +1 through +100 after conditioning).

ii. If a CMV antigen or PCR plasma test is reported as positive (i.e. >100 copies per m1) after achieving ANC >1000 and after day 28, the patient should be treated with Ganciclovir (GCV) at 5 mg/kg IV twice daily for 7 days, followed by GCV maintenance therapy at 5 mg/kg/day IV for 5 days each week per SPP guidelines. During GCV therapy, blood counts must be followed twice weekly, or more frequently if ANC <1500. GCV should be discontinued if the ANC decreases to <1000 for 2 consecutive days and foscarnet should be substituted. GCV should be adjusted for renal function per standard nomogram. Dose reduction starts at a creatinine clearance of <70 ml/min.

iii. If PCR is positive prior to day 28 or if ANC <1000, the patient should be treated with foscarnet. Foscarnet therapy should be initiated with a dose of 60 mg/kg IV twice a day for one week, followed by 90 mg/kg IV once daily for 5 days per week. Once ANC >1000 and after day 28, the patient can be treated with GCV maintenance therapy at 5 mg/kg/day IV daily for 5 days per week. During foscarnet therapy, renal function, serum magnesium, calcium, phosphate, and potassium levels should be monitored daily with daily electrolyte repletion as required. Foscarnet dosing requires daily adjustment for patients with impaired renal function.

6. HHV6 Infection

a. All patients should be monitored for reactivation of latent HHV6 by measurement of plasma viral DNA using PCR. Samples should be sent before conditioning and weekly thereafter until day 100 or until the absolute T cell count is >200 cells/μl, whichever comes last.

b. Any patient who develops confusion or signs of encephalitis, or a patient who is PCR positive on 2 consecutive weekly samples, should undergo cerebrospinal fluid sampling for HHV6 DNA by PCR. Patients with positive result in the CSF should be treated for HHV6 reactivation with ganciclovir or foscarnet, as described above.

7. Adenovirus Infection

a. All patients should be monitored weekly for adenovirus infection by measurement of plasma viral DNA using PCR.

b. Stool samples should be sent weekly for adenovirus PCR.

c. Patients with positive PCR in plasma or stool should be treated with cidofovir to prevent disseminated adenoviral infection. The infectious disease service should be consulted regarding administration of cidofovir and probenacid in PCR positive patients.

8. Monitoring for EBV Lymphoproliferative Syndrome:

a. All patients should be monitored weekly for EBV activation by plasma PCR. Weekly monitoring should continue until day 100 or until absolute T cell count is >200 cells/μl.

b. Pre-emptive therapy of EBV-LPS with the anti-B cell chimeric monoclonal antibody, Rituximab will follow the rules in the table below:

Rules for Pre-emptive Therapy:

| EBV-PCQ | Clinical features of | Management | |
|---|---|---|---|
| (copies/ml) | EBV-PTLD* | Staging | Treatment |
| <1000 | 0 | No | No |
| >100-<1000 | ≧1 | Yes (9.842) | No |
| ≧1000 | 0 | No | Yes |
| (on 2 consecutive | | | |
| testings) | | | |
| ≧1000 | ≧1 | Yes | Yes |

*FUO ≧ 39° C., Lymphadenopathy, hepatosplenomegaly

c. Staging—At commencement of pre-emptive therapy a CT scan of the chest and abdomen should be performed. Tissue diagnosis which includes EBER and LMP-1 immunocytochemistry should be attempted for any mass or lesion that appears suspicious for a lymphoproliferative process. Other diagnostic or staging studies should be performed as clinically indicated.

d. Treatment with Rituximab: Rituximab should be administered at a dose of 375 mg/m2 over 3-12 hours IV within 24 h of intention to treat. Pre-treatment send 10 cc of blood into EDTA-tube for confirmatory EBV-PCR and quantitation of circulating B cells and 10 cc into a clotted tube for serum IgG, IgA and IgM levels. Pre-medicate patient with acetaminophen and diphenhydramine. In patients with biopsy proven EBV-LPS, or clinical and radiological evidence suggestive of EBV-LPS and >1000 copies per ml an attempt will be made to reduce concomitant immunosuppression. Additional therapies to treat EBV-LPS such as conventional cytotoxic agents should be considered. Response to Rituximab will be assessed. In the event of falling titer or negative EBV-PCQ no further doses of Rituximab will be administered. If the titer is stable or rising a maximum of 4 subsequent weekly doses may be given.

### G. THERAPEUTIC ANTIBODIES AND USE THEREOF

A subject afflicted with an autoimmune or alloimmune disease or an infection as described herein may be administered therapeutic antibodies (e.g., human monoclonal antibodies, or chimeric antibodies containing a human immunoglobulin constant region) to treat the disease.

In general, the therapeutic antibodies may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a liquid and is preferably formulated with the compound as a unit-dose formulation which may contain from 0.01 or 0.5% to 95% or 99% by weight of the active compound.

The therapeutic antibodies may be administered to the subject by any medically appropriate procedure, e.g., normal intravenous or intra-arterial administration, injection into the cerebrospinal fluid).

Dosage of the therapeutic antibody will likewise depend, among other things, the condition of the subject, the particular category or type of disorder being treated, the route of administration, the nature of the therapeutic agent employed, and the sensitivity of the disorder to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

In some embodiments, blocking antibodies can be administered prior to the therapeutic antibodies in like manner as described in U.S. Pat. No. Re 38,008 to Abrams.

The present invention is explained in greater detail in the following non-limiting Examples.

### EXPERIMENTAL

Abbreviations used herein: β₂-m, β₂-microglobulin; ER, endoplasmic reticulum; Endo H, Endoglycosidase H; Treg, CD25+ CD4+ regulatory T cells.

Cell preparations. Peripheral blood was obtained from healthy donors, and peripheral blood mononuclear cells (PBMCs) were isolated by Ficoll-Hypaque. Positive selection for T, B or monocyte cells was performed using CD3−, CD19− or CD14− MicroBeads, an LS+/VS+ column and MidiMACS separator (Miltenyi Biotec). CD3+ CD8+ and CD3+ CD4+ cells were further selected from CD3+ populations using CD8− and CD4− Microbeads. CD56+ CD3− cells were isolated from PBMCs by two-step immunomagnetic cell sorting. T cells were depleted using mAb against CD3 and then NK cells were enriched from the depleted cell fraction using mAb against CD56.

Cell cultures. Isolated CD3+CD8+ or CD3+CD4+ cells were cultured in RPMI1640-Hepes containing 10% pooled human serum, 50 uM β-mercaptoethanol, 5 U/ml IL-2, 10 ng/ml PMA and 1 uM ionomycin. CD3+ CD8+ T cell clones (kindly provided by S. R. Riddell) were expanded in RPMI1640-Hepes containing 10% pooled human serum and 50 uM β-mercaptoethanol, with irradiated 500×LCL and 100×PBMC as feeders and 30 ng/ml OKT3. IL2 at 50 U/ml was added the next day and fresh medium without OKT3 but with IL-2 was added at day 5. Cells were fed again at day 8 and day 11. To rest T cell clones, cells were washed twice at day 14, cultured in wells of 12-well plates with T cell clones and PBMC feeder cell numbers at 4×10⁶ and 1×10⁶ respectively, in RPMI1640-Hepes containing 10% pooled human serum and 50 μM β-mercaptoethanol.

CD19+ cells were stimulated as described previously (27). Briefly, CD40 ligand (CD40L) transfected NIH 3T3 cells were irradiated at 9600 rads, plated out at 8×10⁶ cells per well of 6-well plates. Positive selected CD19+ cells were suspended in Iscoves with 10% pooled human serum, 50 ug/ml transferring, 5 ug/ml insulin, 15 ug/ml gentamicin, 3.4 ng/ml IL-4 and 0.66 ug/ml CSA at 2×10⁶ cells/ml.

To stimulate CD56+CD3− cells, two step immunomagnetic sorted cells were cultured in RPMI1640 supplemented with 10% human serum and 25 ng/ml of IL-12 at 50×10⁴ cells/ml. CD14+ cells were activated in 100 ng/ml of LPS in RPMI-10% pooled human serum at 2-4×10⁶ cells/ml. PBMCs obtained from TAP or Tapasin mutant patients were cultured as for CD3+CD4+ cells from normal individuals.

To generate dendritic cells (DCs), monocytes were isolated from PBMC by panning adherent cells on a tissue culture dish in RPMI 1640-10% FCS at 37° C., 5% CO₂ for 90 min, and culturing in RPMI1640 containing 10% FCS, GM-CSF (Amgen) 800 U/ml and IL-4 (R&D) 1000 U/ml at 3×10⁵ cells/ml. Cultures were fed at day 3 and immature DCs were induced to mature DCs by adding 5 ng/ml IL-1β (R&D), 25 ng/ml TNF-α (R&D), 10 ng/ml IL-6 (R&D) and 1 ug/ml PGE2 into culture medium at day 6. Mature DCs were harvested at 24 hr later. To set up stimulation of primary antigen specific T cells (28), mature DCs were induced as above from PBMCs isolated from a CMV+ individual expressing HLA-A2. Cells were preload with 10 ug/ml of pp65 peptide for 4 hr, washed once, irradiated at 3000 rad and mixed with PBMC obtained from the same donor at a 1:20 ratio in RPMI1640, 10% pooled human serum. 5 U/ml of IL-2 was added at days 1 and 3.

Immunofluorescence staining and FACS analysis. MAbs 3D11, 4A11 and 3D12 were employed in indirect immunofluorescence staining as previously described (23). Briefly, cells were incubated with saturating concentration of primary Abs, followed by washing and labeling with FITC- or PE-conjugated goat F(ab′)₂ anti-mouse Ig (BioSource, Camarillo, Calif.). As indicated in certain experiments, biotinylated Abs at 10 ug/ml were combined with CD3-FITC and CD20-PE and detected with streptavidin-Cy-Chrome at 1:50 dilutions. Samples were analyzed on a FACScan cytometer (Becton Dickinson, Mountain View, Calif.).

Cell Surface Biotinylation, Immunoprecipitation and Endo-H digestion. Cell surface proteins of CD3+CD8+ T cell clones, B-LCL and monocyte cells were labeled by Sulfo-NHS-LC-biotin (pierce), immunoprecipitated with streptavidin-agarose beads as described previously (23) with some changes. Briefly, activated CD3+CD8+ cells were collected, washed and biotinylated with Sulfo-NHS-LC-biotin (Pierce) (300 ng/ml) at room temperature for 30 min. After extensive washing with ice cold PBS, cells were lysed at 20×10⁶ cells per ml of lysis buffer containing 10 mM Tris, pH 7.8, 140 mM NaCl, 1% TritonX-100, 200 uM PMSF, 10 ug/ml papstalin and 14 ug/ml aprotinin for an hour on ice. Cell surface proteins were precipitated with 50% streptavidin-agrose (Pierce) overnight at 4° C. The streptavidin-agarose beads were separated from the cell lysate by centrifugation, washed five times in 10 mM Tris-HCl, pH7.5, 0.05% CHAPS, 0.1% SDS and 300 mM NaCl, one time in 0.5% NP-40 in PBS. Endo H (Roche) digestion was carried out according to manufacturer's suggestion.

Western Blotting and Immunodetection. Cell surface and total cell proteins were separated on an 11% SDS-PAGE gel and electroblotted as described (17, 23). HLA-F protein was detected by mAb 3D11 followed by horseradish peroxidase-labeled goat anti-mouse Ig's (BioSource) at 1:5,000 dilutions, and visualized with an enhanced chemiluminescence system (ECL. Amersham, Arlington Heights, Ill.).

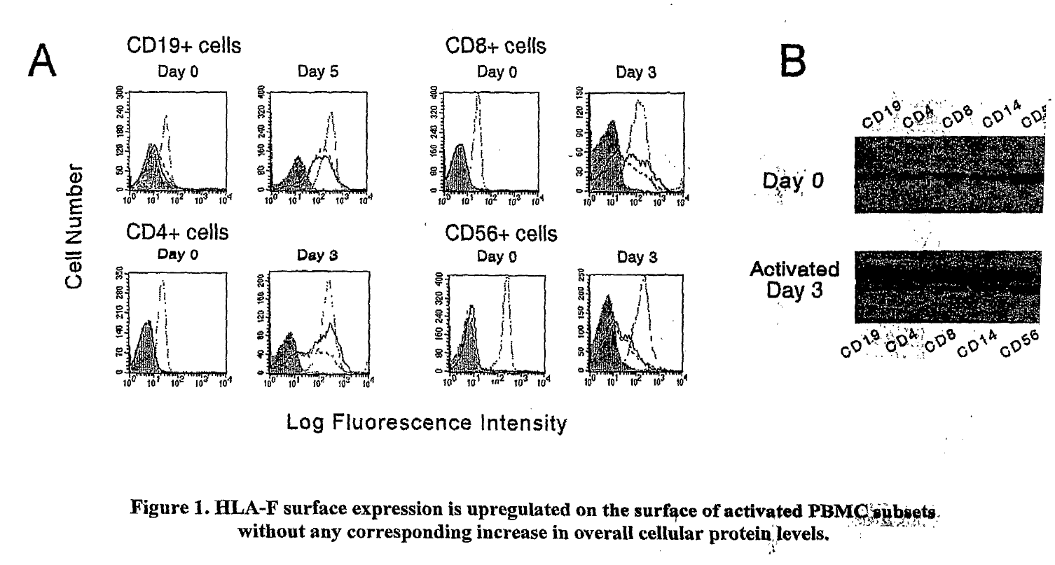

Results and Discussion. In previous work, the only demonstrations of HLA-F surface expression were described in two reports from this lab (16, 23). Although other studies did not detect surface expression (21, 22), one constant among all the studies was the finding that while HLA-F was expressed within at least some peripheral blood lymphocytes as evidenced by western analysis, there was no surface expression observed (21-23) and an intracellular role for HLA-F was hypothesized (22). However, the fact that our antibodies were able to detect HLA-F on the surface of lymphoblastoid cell lines suggested that since LCL share some proliferation and differentiation characteristics with activated B cells (26), expression might be upregulated in activated cells. Therefore we examined the expression of HLA-F in vivo under conditions that favored activation of immune cells, examining separately monocytes, B, T, and NK cells before and after activation (FIG. 1). Whereas there was no surface expression on any cell subset of peripheral blood lymphocytes for each of the T cell, B cell, and NK cell subsets, high levels of HLA-F could be observed within days of activation. Interestingly, there was no concomitant increase in total HLA-F protein upon activation in any of the cell subsets as similar quantities were present before and after activation in all cells examined (FIG. 1B). Identical results were obtained using either of two distinctly reactive anti-F reagents, 3D11 and 4A11.

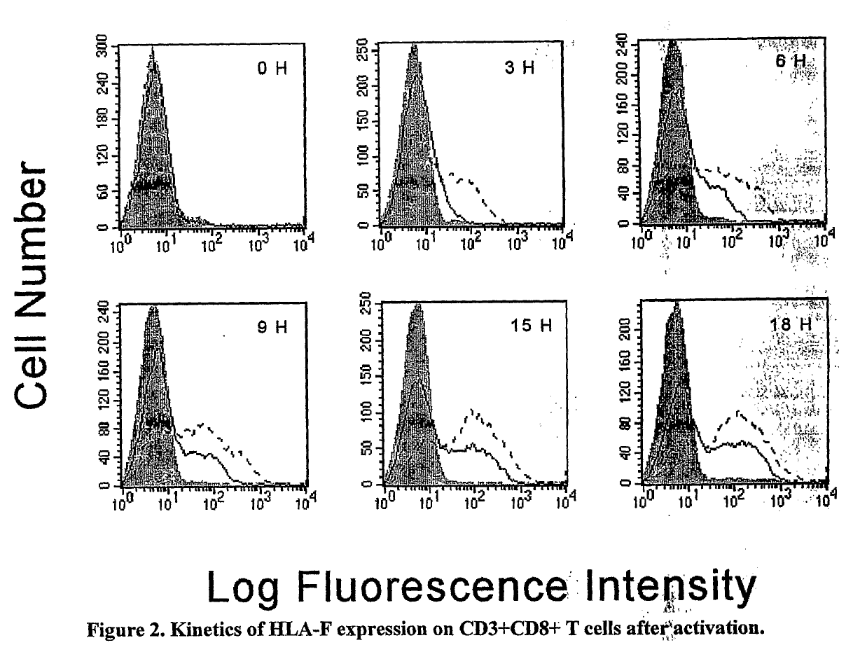

Because it was apparent that HLA-F protein was present in lymphocytes before activation, we next examined the timing of HLA-F surface transport on T cells as a first step towards understanding factors that might control surface expression. This experiment showed that HLA-F surface expression was upregulated as early as 3 hours after T cell activation, correlated with and slightly preceding initiation of DNA synthesis in activated cells (29, 30) (FIG. 2). Surface expression also preceded the appearance of the previously described two transcripts for CD25, which are significantly upregulated by 6 hours after activation (31). The transport of HLA-F to the surface soon after activation is consistent with our hypothesis suggested below that HLA-F is a marker for the activated immune response communicating some aspect of that activation to regulatory immune cells.

One interesting aspect of early expression was the differential binding of the two HLA-F specific antibodies used. Anti-F reagent 3D11 detects protein earlier than antibody 4A11, although at later times binding of the two antibodies is essentially equivalent (FIG. 1A). We have mapped the epitopes of these antibodies to distinct sites on the protein using mutagenesis and found 3D11 binding to be dependent on residues in the alpha 2 domain while 4A11 binding depends on residues in the alpha 1 protein domain (data not shown). Previous work showed that surface HLA-F protein may be only partially complexed with β₂-microglobulin and that some may be present as free heavy chain (23). Although our previous studies indicated that both antibodies do react with both free heavy chain and HLA-F complexed with β₂-m, differential reactivity with these forms is a possibility. This would in turn suggest that different quantities of alternative HLA-F protein structures are present on the surface early in T cell activation.

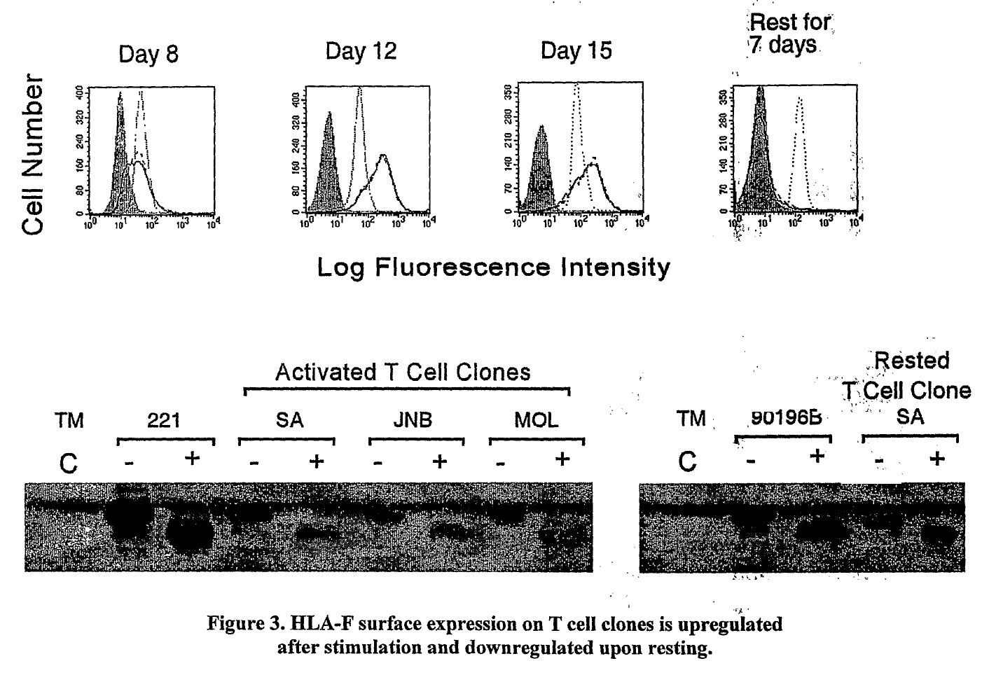

In order to examine the HLA-F protein and to compare the expression of HLA-F on normal cells with previous work on cell lines, we examined HLA-F protein in T cell clones. Two observations were noteworthy. As expected HLA-F expression was upregulated soon after a clone was activated and peaked at day 15 after activation was initiated (FIG. 3). After resting for seven days, no HLA-F expression was observed, coincident with the ceasing of active cell division. In addition, nearly all of the HLA-F present on the surface of resting or non-activated T cells was Endo H sensitive. In B and monocytes cell lines we previously showed that some surface HLA-F was present as an Endo H resistant form similar to other MHC class I proteins. However, a similar examination of HLA-F on the surface of activated T cell clones demonstrated that most or all of the protein was in fact present as an Endo H sensitive form (FIG. 3, lower), further suggesting that HLA-F may proceed through a pathway distinct from other MHC class I proteins on its way to the cell surface. For classical MHC class I and II molecules, the routes and loading compartments are essential for binding to and presenting cytosolic and endosomal antigens, respectively. Other pathways for intracellular trafficking routes are distinct from these MHC proteins including those followed by CD1 molecules (32). In that regard, it is interesting to note that the HLA-F protein cytoplasmic tail, which is modified relative to classical class I due to exclusion of the exon seven sequences from the mature mRNA (9), shares key residues with the CD1d cytoplasmic tail. These residues are part of a signaling mechanism directing interaction the adaptor-3 protein complexes that mediate the endosomal localization of CD1d (33).

As an MHC class I molecule, understanding the factors that control expression of HLA-F requires an investigation of the interaction or dependency on the peptide transporters TAP and Tapasin. Although there is no evidence yet that HLA-F normally binds peptide, an examination of HLA-F expression in TAP negative and Tapasin negative B-LCLs did show a partial dependence of HLA-F surface expression on Tapasin, specifically for the Endo H resistant HLA-F protein. While HLA-F may physically interact with TAP (22), it may not depend on TAP for surface expression (23). Surface expression on EBV transformed lymphoblastoid cell lines and a subset of monocyte cell lines correlated with the presence of a limited amount N-glycosylated HLA-F being present. No difference in overall levels of HLA-F was observed on parent LCL 45.1, TAP negative 0.134, or TAP restored 0.134 line 2C2 (23), no change in the quantity of Endo H resistant material was apparent. There did, however, appear to be Tapasin dependence in that a partial reduction in surface levels and a complete elimination of Endo H resistant protein was apparent in Tapasin mutant line 0.220.

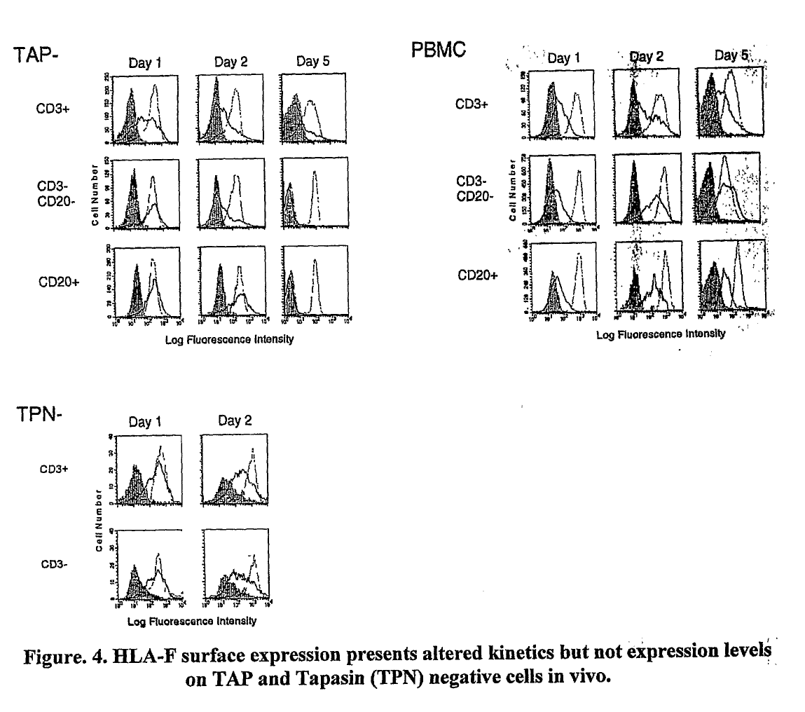

In order to examine in vivo expression of HLA-F in this regard, we took advantage of two individuals that are homozygous for null mutations in each of the TAP and Tapasin genes (34, 35). Using chemical methods for activation of lymphocytes, upregulation of HLA-F was apparent on TAP-CD3+ cells on day one after activation, while cells from a normal individual required up to 5 days before full expression levels were apparent (FIG. 4). In fact, active expression of HLA-F on all three cell types examined was upregulated far sooner than on normal cells peaking on day one on the former versus up to day 5 on the latter. It was not clear why HLA-F expression was downregulated about day 5 soon after activation while cells were still actively expanding. This stands in contrast to our studies with normal cells where cell division was strongly correlated with HLA-F surface expression. Cell division in the TAP negative stimulation continued well beyond day one as cell counts continued to increase up to day 5 (data not shown). An essentially similar picture emerged when Tapasin PBMC was examined, again with HLA-F expression peaking at day one, and with levels beginning to subside as soon as day 2. It was not possible to examine cells beyond this timepoint due to the small number of cells available and the poor expansion upon activation.

How the altered kinetics of HLA-F expression might affect a normal immune response is not clear from the disease pathogenesis of these two homozygous mutant types. TAP-deficient patients have serious immune related symptoms including respiratory inflammations and skin ulcers (34). In one study a mutation in the TAP2 gene responsible for the defective expression of the TAP complex resulted in the presence of autoreactive natural killer (NK) cells and gammadelta T lymphocytes in the peripheral blood cells of two patients (36). However, the Tapasin-deficient subject studied in this report has not shown any similar symptoms, or any apparent immune deficiency at all. A more careful examination of HLA-F expression and its potential effect on immune monitoring in these individuals is required before any conclusions about their altered HLA-F expression can be made. Since HLA-F expression is altered in a similar manner in both, it may suggest a common effect on the mechanisms controlling HLA-F expression. These facts will need to be reconciled with other features of HLA-F intracellular transport as they come to light.

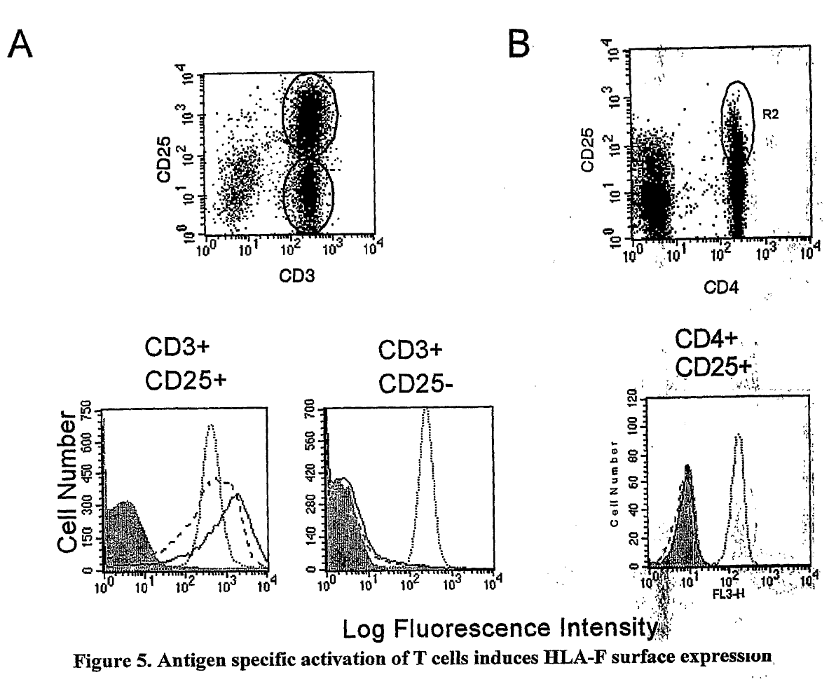

Although mitogen and antibody mediated lymphocyte activation was able to induce surface expression, a more precise activation response was examined by using a CMV peptide specific response. When PBMC isolated from a CMV+ individual was stimulated by mature dendritic cells loaded with CMV peptide pp65, stimulated and expanded memory T cells were assayed by staining with anti-CD25. As can be seen in FIG. 5A, all of the activated CD25+ T cells strongly expressed surface HLA-F while other CD25− T cells retained the HLA-F null phenotype. This demonstrated that all activation methods tested, including one that paralleled an in vivo response, do yield high levels of surface HLA-F specific to activated lymphocytes. Further, it is apparent that HLA-F is expressed only on activated CD4 and CD8 T cells and not on CD4+ CD25+ regulatory T cells (FIG. 5B). This important difference with respect to CD25 expression is suggestive not only of potential function for HLA-F but also of improvements to methods and strategies that now employ CD25 as an activation marker to select against activated cells. (37-39).

### Example 2

### Depletion Experiments

For depletion experiments, cells were cultured with alloantigen for 5 or 7 days, washed, labeled with biotinylated 3D11, 4A11 or IgG1 control antibody and positive cells were removed using Streptavidin MicroBeads and LS+/VS+ column (Miltenyi Biotec). Negative cells were recultured in medium without stimulators for an additional 5 or 4 days, harvested on day 10 and then restimulated. Primed cells were restimulated with fresh stimulator cells from the original donor or from an unrelated donor in 96-well plates. 10 replicate plates were set up and one was harvested each day for 10 consecutive days.

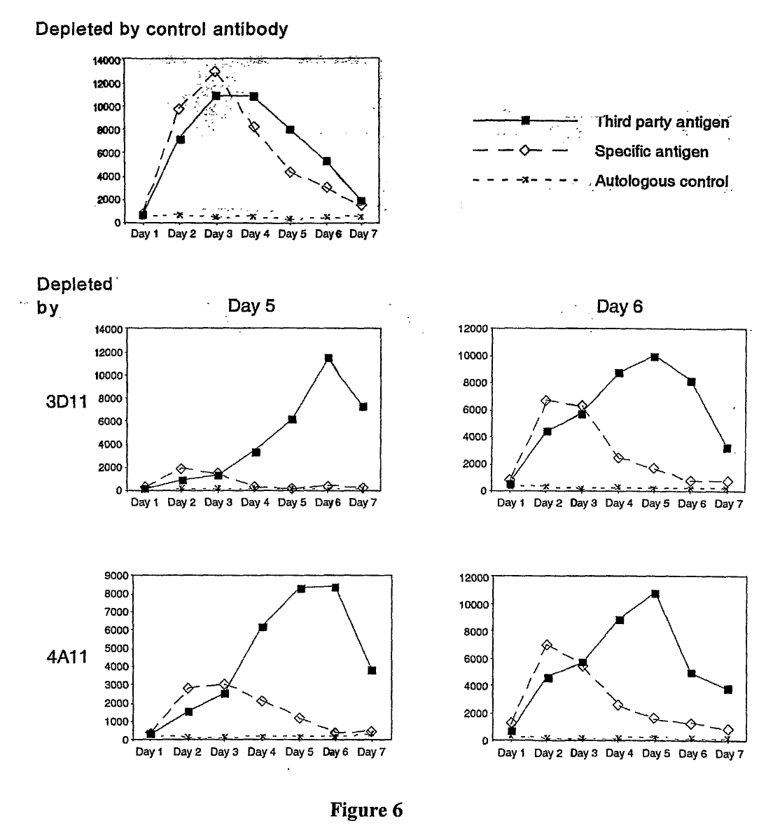

As shown in FIG. 6, Depletion of HLA-F expressing cells after primary MLR inhibits secondary MLR without compromising third party reactivity. A standard MLR against 2^{nd} party cells was depleted at various days during a primary MLR after stimulation with 3D11, 4A11, or to control antibody bound to magnetic beads. The cells remaining were recultured in medim until day 10 when then treated with either specific antigen from the same 2^{nd} party or with 3^{rd} party antigen contributed by other donor cells. Representative tritium incorporation results for each anti-HLA-F antibody from day 5 and 7 and from control antibody are shown.

### Example 3

### Non-Human Primate HLA-F Staining

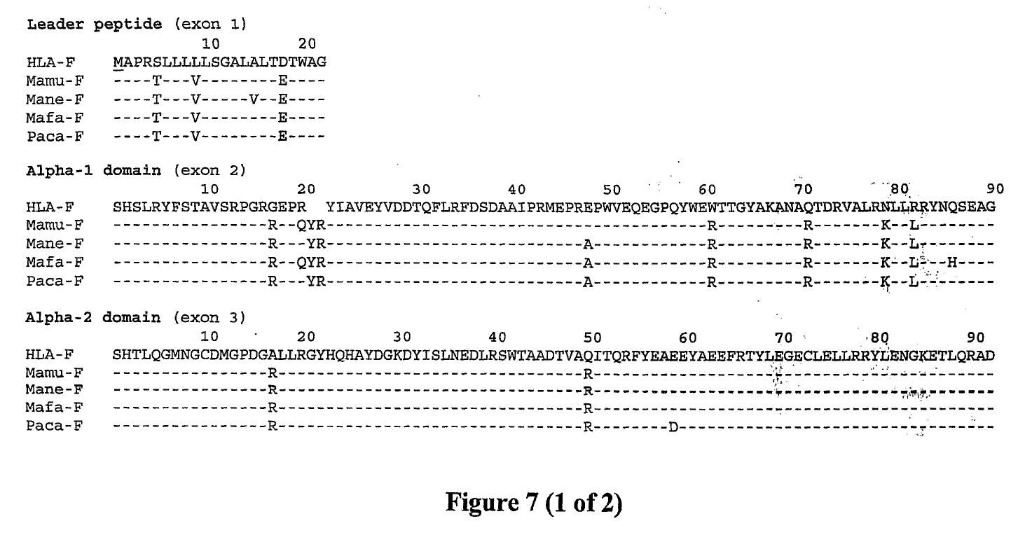

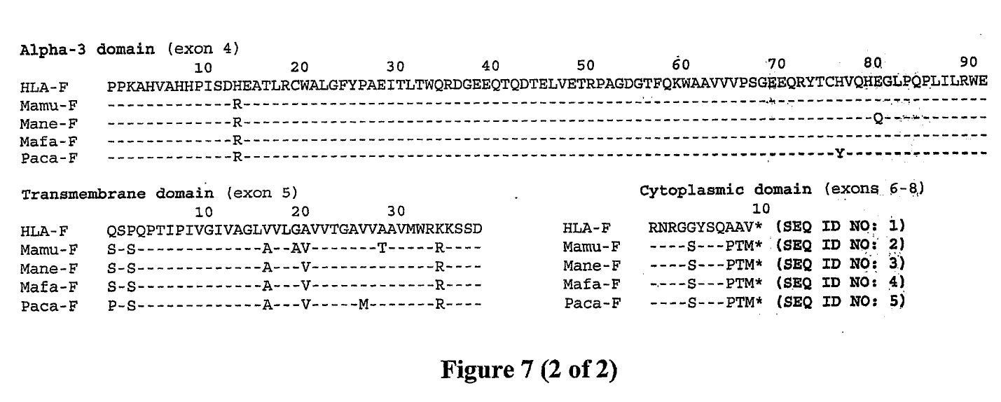

FIG. 7 shows the amino acid sequences of human HLA-F and non-human primate homologous proteins. Sequences were derived from RT-PCR analysis of mRNA isolated from PBMC from each species. Primers were designed based on the previously established mamu-F sequence and sequence analysis was carried out using standard methods. Identity to the human HLA-F sequence is indicated by a dash. The start methionine corresponding to the ATG initiation codon in the human HLA-F cDNA sequence has been underlined. The asterisk at the end of the amino acid sequences indicates the termination codon in the respective cDNA sequences. The various domains of the HLA-F equivalent proteins are aligned separately. The numbering above the human HLA-F amino acid sequence is according to convention.

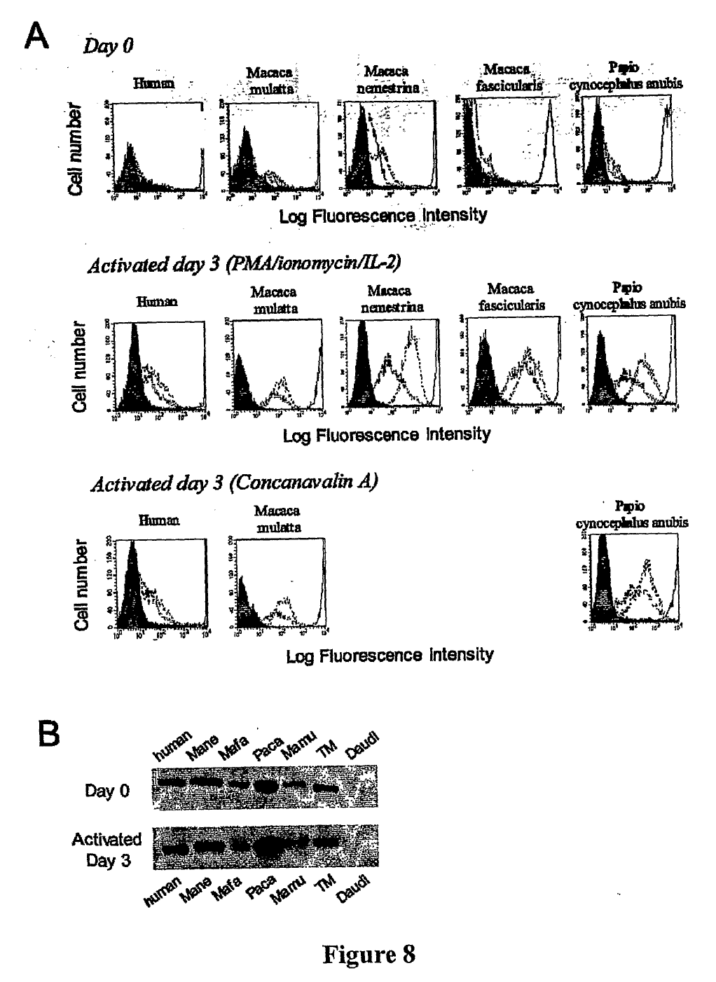

FIG. 8 shows that HLA-F equivalent proteins found in 4 nonhuman primates (NHP) are reactive with human anti-HLA-F antibodies and expression patterns of NHP-F proteins parallel those found in humans. A) HLA-F and equivalents expression on the surface of PBMC of human and different species of non-human primates at day 0 and day 3 after stimulation with either PMA/ionomycin/IL-2 or Concanavalin A as indicated. FACS profiles were generated with the following antibodies: Anti-F antibodies 4A11 (dashed line) and 3D11 (dotted line), the mouse IgG1 antibody as a negative control (shaded histogram) and the pan-class-I antibody W6/32 as a positive control (solid line). B) Western analysis of PBMC of human and different species of non-human primates at day 0 and day 3 after stimulation with PMA/ionomycin/IL-2.

### REFERENCES

1. Townsend, A., and H. Bodmer. 1989. Antigen recognition by class I-restricted T lymphocytes. Annu Rev Immunol 7:601.
2. Accolla, R. S. et al., 1995. MHC: orchestrating the immune response. Immunol Today 16:8.
3. Lanier, L. L., and J. H. Phillips. 1995. NK cell recognition of major histocompatibility complex class I molecules. Semin Immunol 7:75.
4. Colonna, M. 1996. Natural killer cell receptors specific for MHC class I molecules. Curr Opin Immunol 8:101.
5. Spies, T., V. et al., 1992. Presentation of viral antigen by MHC class I molecules is dependent on a putative peptide transporter heterodimer. Nature 355:644.
6. Spies, T., and R. DeMars. 1991. Restored expression of major histocompatibility class I molecules by gene transfer of a putative peptide transporter. Nature 351:323.
7. Geraghty, D. E. et al., 1987. A human major histocompatibility complex class I gene that encodes a protein with a shortened cytoplasmic segment. Proc Natl Acad Sci USA 84:9145.
8. Koller, B. H. et al., 988. HLA-E. A novel HLA class I gene expressed in resting T lymphocytes. J Immunol 141:897.
9. Geraghty, D. E. et al., 1990. Human leukocyte antigen F (HLA-F). An expressed HLA gene composed of a class I coding sequence linked to a novel transcribed repetitive element. J Exp Med 171:1.
10. Geraghty, D. E. 1993. Structure of the HLA class I region and expression of its resident genes. Curr Opin Immunol 5:3.
11. Lee, N. et al., 1998. HLA-E is a major ligand for the natural killer inhibitory receptor CD94/NKG2A. Proc Natl Acad Sci USA 95:5199.
12. Kovats, S. et al., 1990. A class I antigen, HLA-G, expressed in human trophoblasts. Science 248:220.
13. Ishitani, A., and D. E. Geraghty. 1992. Alternative splicing of HLA-G transcripts yields proteins with primary structures resembling both class I and class II antigens. Proc Natl Acad Sci USA 89:3947.
14. Le Bouteiller, P. et al., 1996. Placental expression of HLA class I genes. Am J Reprod Immunol 35:216.
15. Lee, N. et al., 1995. The membrane-bound and soluble forms of HLA-G bind identical sets of endogenous peptides but differ with respect to TAP association. Immunity 3:591.
16. Ishitani, A. et al., 2003. Protein expression and peptide binding suggest unique and interacting functional roles for HLA-E, F, and G in maternal-placental immune recognition. J Immunol 171:1376.
17. Lee, N. et al., 1998. HLA-E surface expression depends on binding of TAP-dependent peptides derived from certain HLA class I signal sequences. J Immunol 160:4951.
18. Shiroishi, M. et al., 2003. Human inhibitory receptors Ig-like transcript 2 (ILT2) and ILT4 compete with CD8 for MHC class I binding and bind preferentially to HLA-G. Proc Natl Acad Sci USA 100:8856.
19. Kaiser, B. K. et al., 2005. Interactions between NKG2x immunoreceptors and HLA-E ligands display overlapping affinities and thermodynamics. J Immunol 174:2878.
20. Llano, M. et al., 1998. HLA-E-bound peptides influence recognition by inhibitory and triggering CD94/NKG2 receptors: preferential response to an HLA-G-derived nonamer. Eur J Immunol 28:2854.
21. Lepin, E. J. et al., 2000. Functional characterization of HLA-F and binding of HLA-F tetramers to ILT2 and ILT4 receptors. Eur J Immunol 30:3552.
22. Wainwright, S. D. et al., 2000. HLA-F is a predominantly empty, intracellular, TAP-associated MHC class Ib protein with a restricted expression pattern. J Immunol 164:319.
23. Lee, N., and D. E. Geraghty. 2003. HLA-F surface expression on B cell and monocyte cell lines is partially independent from tapasin and completely independent from TAP. J Immunol 171:5264.
24. Daza-Vamenta, R. et al., 2004. Genetic divergence of the rhesus macaque major histocompatibility complex. Genome Res 14:1501.
25. O'Callaghan, C. A., and J. I. Bell. 1998. Structure and function of the human MHC class Ib molecules HLA-E, HLA-F and HLA-G. Immunol Rev 163:129.
26. O'Nions, J., and M. J. Allday. 2004. Proliferation and differentiation in isogenic populations of peripheral B cells activated by Epstein-Barr virus or T cell-derived mitogens. J Gen Virol 85:881.
27. Kondo, E. et al., 2002. Efficient generation of antigen-specific cytotoxic T cells using retrovirally transduced CD40-activated B cells. J Immunol 169:2164.
28. Bonini, C. et al., 2001. Targeting antigen in mature dendritic cells for simultaneous stimulation of CD4+ and CD8+ T cells. J Immunol 166:5250.
29. Wang, X. et al., 1998. Role of proteasomes in T cell activation and proliferation. J Immunol 160:788.
30. Ertesvag, A. et al., 2002. Retinoic acid stimulates the cell cycle machinery in normal T cells: involvement of retinoic acid receptor-mediated IL-2 secretion. J Immunol 169:5555.
31. Woerly, G. et al., 1996. Effect of rapamycin on the expression of the IL-2 receptor (CD25). Clin Exp Immunol 103:322.
32. Brigl, M., and M. B. Brenner. 2004. CD1: antigen presentation and T cell function. Annu Rev Immunol 22:817.
33. Lawton, A. P. et al., 2005. The mouse CD1d cytoplasmic tail mediates CD1d trafficking and antigen presentation by adaptor protein 3-dependent and -independent mechanisms. J Immunol 174:3179.
34. Furukawa, H. et al., 1999. Cell surface expression of HLA-E molecules on PBMC from a TAP 1-deficient patient. Tissue Antigens 53:292.
35. Yabe, T. et al., 2002. A subject with a novel type I bare lymphocyte syndrome has tapasin deficiency due to deletion of 4 exons by Alu-mediated recombination. Blood 100:1496.
36. Moins-Teisserenc, H. T. et al., 1999. Association of a syndrome resembling Wegener's granulomatosis with low surface expression of HLA class-I molecules. Lancet 354:1598.
37. Swiatecka-Urban, A. 2003. Anti-interleukin-2 receptor antibodies for the prevention of rejection in pediatric renal transplant patients: current status. Paediatr Drugs 5:699.
38. Herwartz, C. et al., 1992. Rejection prophylaxis with interleukin-2 receptor antibody BT 563: mechanisms of action on human cells. Transpl Int 5 Suppl 1:S684.
39. Wiland, A. M., and B. Philosophe. 2004. Daclizumab induction in solid organ transplantation. Expert Opin Biol Ther 4:729.
40. Piccirillo, C. A., and E. M. Shevach. 2004. Naturally-occurring CD4+CD25+ immunoregulatory T cells: central players in the arena of peripheral tolerance. Semin Immunol 16:81.
41. Shevach, E. M. 2000. Regulatory T cells in autoimmmunity*. Annu Rev Immunol 18:423.
42. Sasaki, Y. et al., 2004. Decidual and peripheral blood CD4+CD25+ regulatory T cells in early pregnancy subjects and spontaneous abortion cases. Mol Hum Reprod 10:347.
43. Heikkinen, J. et al., 2004. Phenotypic characterization of regulatory T cells in the human decidua. Clin Exp Immunol 136:373.
44. Izcue, A., and F. Powrie. 2005. Prenatal tolerance—a role for regulatory T cells? Eur J Immunol 35:379.
45. Saito, S., Y. Sasaki, and M. Sakai. 2005. CD4(+)CD25(high) regulatory T cells in human pregnancy. J Reprod Immunol 65:111.
46. Aluvihare, V. R. et al., 2004. Regulatory T cells mediate maternal tolerance to the fetus. Nat Immunol 5:266.
47. Bielekova, B. et al., 2004. Humanized anti-CD25 (daclizumab) inhibits disease activity in multiple sclerosis patients failing to respond to interferon beta. Proc Natl Acad Sci USA 101:8705.
48. Malek, T. R., and A. L. Bayer. 2004. Tolerance, not immunity, crucially depends on IL-2. Nat Rev Immunol 4:665.
49. Martin, P. J. et al., 2004. Evaluation of a CD25-specific immunotoxin for prevention of graft-versus-host disease after unrelated marrow transplantation. Biol Blood Marrow Transplant 10:552.

The foregoing is illustrative of the present invention, and is not to be construed as limiting thereof. The invention is defined by the following claims, with equivalents of the claims to be included therein.

That which is claimed is:

1. A method of detecting an immune response in a mammalian subject in need thereof, comprising:
(a) withdrawing blood or a blood fraction containing immune cells from said subject;
(b) contacting said blood or blood fraction to an antibody that specifically binds to the cell surface HLA-F histocompatibility protein of activated mammalian immune cells, which antibody does not bind to the cell surface of non-activated mammalian immune cells; and then
(c) detecting the presence or absence of binding of said immune cells to said antibody, the presence of binding indicating the presence of an immune response in said subject.

2. The method of claim 1, wherein said immune cells are selected from the group consisting of T-lymphocytes, B-lymphocytes, NK cells, monocytes, and combinations thereof.

3. The method of claim 1, wherein said immune response is caused by an infection.

4. The method of claim 1, wherein said immune response is an autoimmune disease.

5. The method of claim 4, wherein said autoimmune disease is selected from the group consisting of multiple sclerosis, rheumatoid arthritis, juvenile oligoarthritis, type I diabetes mellitus, inflammatory bowel disease, Crohn's disease, scleroderma, psoriasis, atherosclerosis, Hashimoto's thyroiditis, Addison's disease, and systemic lupus erythematosus (SLE).

6. The method of claim 1, wherein said immune response is an allergic response or alloimmune disease.

7. The method of claim 6, wherein said immune response is an alloimmune disease selected from the group consisting of graft versus host disease and tissue transplant rejection, or said disease is an allergic response to venom, animal dander, pollen, fungi or fungal spores, dust mites, food allergens and drugs.

8. The method of claim 1, wherein said antibody is a monoclonal antibody.

9. The method of claim 1, wherein said antibody does not bind to the HLA-A, HLA-B, HLA-C, HLA-E, or HLA-G histocompatibility proteins of activated mammalian T-lymphocytes in either activated or non-activated form.

10. The method of claim 1, wherein said detecting step is carried out by heterogeneous immunoassay.

11. The method of claim 1, wherein said detecting step is carried out by homogeneous immunoassay.

12. A method of treating an undesired immune response in a mammalian subject in need thereof, comprising:
(a) withdrawing blood or a blood fraction containing immune cells from said subject;
(b) contacting said blood or blood fraction to an antibody that specifically binds to the cell surface HLA-F histocompatibility protein of activated mammalian immune cells, which antibody does not bind to the cell surface of non-activated mammalian immune cells;
(c) separating said blood or blood fraction from said antibodies to thereby deplete said blood or blood fraction of activated immune cells; and then
(d) returning said blood or blood fraction to said subject.

13. The method of claim 12, wherein said undesired immune response is selected from the group consisting of autoimmune disease, alloimmune disease, or acquired immune deficiency disease.

14. The method of claim 12, wherein said immune cells are selected from the group consisting of T-lymphocytes, B-lymphocytes, NK cells, monocytes, and combinations thereof.

15. The method of claim 12, wherein said disease is an autoimmune disease.

16. The method of claim 15, wherein said autoimmune disease is selected from the group consisting of multiple sclerosis, rheumatoid arthritis, juvenile oligoarthritis, type I diabetes mellitus, inflammatory bowel disease, Crohn's disease, scleroderma, psoriasis, atherosclerosis, Hashimoto's thyroiditis, Addison's disease, and systemic lupus erythematosus (SLE).

17. The method of claim 12, wherein said disease is caused by an infection.

18. The method of claim 12, wherein said disease is an alloimmune disease.

19. The method of claim 18, wherein said alloimmune disease is selected from the group consisting of graft versus host disease and tissue transplant rejection.

20. The method of claim 12, wherein said antibody is a monoclonal antibody.

21. The method of claim 12, wherein said antibody does not bind to the HLA-A, HLA-B, HLA-C, HLA-E, or HLA-G histocompatibility proteins of activated mammalian T-lymphocytes in either activated or non-activated form.

22. The method of claim 12, wherein said antibody is coupled to a solid support.

23. The method of claim 12, wherein said withdrawing step (a) and said returning step (d) are carried out continuously or in batch form.

24. The method of claim 12, wherein said steps (a) through (d) are carried out by apheresis.

25. A composition comprising a solid support having an antibody coupled thereto, which antibody specifically binds to the cell surface HLA-F histocompatibility protein of activated mammalian T-lymphocytes, and which antibody does not bind to the-cell surface of non-activated mammalian T-lymphocytes.

26. The composition of claim 25, wherein said antibody is a monoclonal antibody.

27. The composition of claim 25, wherein said antibody does not bind to the HLA-A, HLA-B, HLA-C, HLA-E, or HLA-G histocompatibility proteins of activated mammalian T-lymphocytes in either activated or non-activated form.

28. A pharmaceutically acceptable composition comprising a mammalian blood or blood fraction,
said blood or blood fraction comprising blood serum, immune cells, optionally blood platelets, and optionally red blood cells,
and wherein said immune cells (i) consist of non-activated immune cells that do not express HLA-F on the cell surface thereof, and (ii) are depleted of activated immune cells that express HLA-F on the cell surface thereof.

29. The composition of claim 28 depleted of activated immune cells activated by a preselected immunogen.

30. The composition of claim 28, wherein said immune cells are selected from the group consisting of T-lymphocytes, B-lymphocytes, NK cells, monocytes, and combinations thereof.

31. The composition of claim 28 produced by the process of:
(a) withdrawing blood or a blood fraction containing immune cells from a mammalian subject afflicted with an undesired immune response;
(b) contacting said blood or blood fraction to an antibody that specifically binds to the cell surface HLA-F histocompatibility protein of activated mammalian immune cells, which antibody does not bind to the cell surface of non-activated mammalian immune cells;
(c) separating said blood or blood fraction from said antibodies to produce said composition.

32. A method of carrying out an allogenic bone marrow transplant in a subject in need thereof, comprising:
(a) providing a first cell preparation comprising blood stem cells and immune cells;
(b) contacting said first cell preparation to at least one isoantigen from said subject to activate a portion of said immune cells; then
(c) contacting said cell preparation to an antibody that specifically binds to the cell surface HLA-F histocompatibility protein of activated mammalian T-lymphocytes, which antibody does not bind to the cell surface of non-activated mammalian T-lymphocytes; then
(d) separating said cell preparation from said antibodies to produce a depleted cell preparation depleted of activated immune cells; and then
(c) administering said depleted cell preparation to said subject in a bone marrow transplant-effective amount, wherein said cell preparation is depleted of immune cells activated by said isoantigen which would otherwise produce autoantibodies against said subject but contains non-activated immune cells effective for producing an immune response against non-self immunogens by said subject.

33. The method of claim 32, wherein said cell preparation of step (a) is prepared by bone marrow harvest or by apheresis of peripheral stem cells.

34. The method of claim 32, wherein said cell preparation of step (a) is collected from a haploid identical (or “haploidentical”) donor.

35. The method of claim 32, wherein said cell preparation of step (a) is collected from a mismatched unrelated donor.

36. The method of claim 32, wherein said subject is afflicted with leukemia, severe aplastic anemia, lymphoma, multiple myeloma, immune deficiency disorder, or solid tumor cancer.

37. A monoclonal antibody that specifically binds to the cell surface HLA-F histocompatibility protein of activated mammalian T-lymphocytes, and which antibody does not bind to the cell surface of non-activated mammalian T-lymphocytes;
said antibody selected from the group consisting of human monoclonal antibodies and chimeric monoclonal antibodies, said chimeric monoclonal antibodies having a human immunoglobulin constant region.

38. The monoclonal antibody of claim 37 having a cytotoxic group coupled thereto.

39. A pharmaceutical formulation comprising an antibody of claim 37 in a pharmaceutically acceptable carrier.

40. A method of treating an undesired immune response in a subject in need thereof, comprising administering said subject a monoclonal antibody of claim 37 in an amount effective to treat said disease.

41. The method of claim 40, wherein said undesired immune response is an autoimmune disease.

42. The method of claim 41, wherein said undesired immune response is an autoimmune disease selected from the group consisting of multiple sclerosis, rheumatoid arthritis, juvenile oligoarthritis, type I diabetes mellitus, inflammatory bowel disease, Crohn's disease, scleroderma, psoriasis, atherosclerosis, Hashimoto's thyroiditis, Addison's disease, and systemic lupus erythematosus (SLE).

43. The method of claim 40, wherein said undesired immune response is an alloimmune disease.

44. The method of claim 43, wherein said alloimmune disease is selected from the group consisting of graft versus host disease and tissue transplant rejection.

45. The method of claim 40, wherein said undesired immune response is caused by an infection.


SEQUENCE LISTING (5 sequences)

SEQ ID NO: 1 - protein, 346 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGALLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPSGEEQRYTCHVQHEGLPQPLILRWEQSPQPTIPIVGIVAGLVVLGAVVTGAVVAAVMWRKKSSDRNRGGYSQAAV

SEQ ID NO: 2 - protein, 348 aa, Macaca mulatta
MAPRTLLLVLSGALALTETWAGSHSLRYFSTAVSRPGRREPQYRYIAVEYVDDTQFLRFDSDAAIPRMEPREPWVEQEGPQYWERTTGYAKANARTDRVALRKLLLRYNQSEAGSHTLQGMNGCDMGPDGRLLRGYHQHAYDGKDYISLNEDLRSWTAADTVARITQRFYEAEEYAEEFRTYLEGECLELLRRYLENGKETLQRADPPKAHVAHHPISDREATLRCWALGFYPAEITLTWQRDGEEQTQDTELVETRPAGDGTFQKWAAVVVPSGEEQRYTCHVQHEGLPQPLILRWESSSQPTIPIVGIVAGLAVLAVVVTGAVVTAVMWRRKSSDRNRGSYSQPTM

SEQ ID NO: 3 - protein, 348 aa, Macaca nemestrina
MAPRTLLLVLSGALVLTETWAGSHSLRYFSTAVSRPGRREPRYRYIAVEYVDDTQFLRFDSDAAIPRMEPRAPWVEQEGPQYWERTTGYAKANARTDRVALRKLLLRYNQSEAGSHTLQGMNGCDMGPDGRLLRGYHQHAYDGKDYISLNEDLRSWTAADTVARITQRFYEAEEYAEEFRTYLEGECLELLRRYLENGKETLQRADPPKAHVAHHPISDREATLRCWALGFYPAEITLTWQRDGEEQTQDTELVETRPAGDGTFQKWAAVVVPSGEEQRYTCHVQHQGLPQPLILRWESSSQPTIPIVGIVAGLAVLGVVVTGAVVAAVMWRRKSSDRNRGSYSQPTM

SEQ ID NO: 4 - protein, 348 aa, Macaca fascicularis
MAPRTLLLVLSGALALTETWAGSHSLRYFSTAVSRPGRREPQYRYIAVEYVDDTQFLRFDSDAAIPRMEPRAPWVEQEGPQYWERTTGYAKANARTDRVALRKLLLRYNHSEAGSHTLQGMNGCDMGPDGRLLRGYHQHAYDGKDYISLNEDLRSWTAADTVARITQRFYEAEEYAEEFRTYLEGECLELLRRYLENGKETLQRADPPKAHVAHHPISDREATLRCWALGFYPAEITLTWQRDGEEQTQDTELVETRPAGDGTFQKWAAVVVPSGEEQRYTCHVQHEGLPQPLILRWESSSQPTIPIVGIVAGLAVLGVVVTGAVVAAVMWRRKSSDRNRGSYSQPTM

SEQ ID NO: 5 - protein, 348 aa, Papio cynocephalus anubis
MAPRTLLLVLSGALALTETWAGSHSLRYFSTAVSRPGRREPRYRYIAVEYVDDTQFLRFDSDAAIPRMEPRAPWVEQEGPQYWERTTGYAKANARTDRVALRKLLLRYNQSEAGSHTLQGMNGCDMGPDGRLLRGYHQHAYDGKDYISLNEDLRSWTAADTVARITQRFYEADEYAEEFRTYLEGECLELLRRYLENGKETLQRADPPKAHVAHHPISDREATLRCWALGFYPAEITLTWQRDGEEQTQDTELVETRPAGDGTFQKWAAVVVPSGEEQRYTCYVQHEGLPQPLILRWEPSSQPTIPIVGIVAGLAVLGVVVTGAMVAAVMWRRKSSDRNRGSYSQPTM